(19) United States
(12) Patent Application Publication
(10) Pub. No.: US 2025/0275538 A1
(43) Pub. Date: Sep. 4, 2025

(54) COMPOSITIONS CONTAINING L-CARNITINE AND SEAWEED EXTRACT FOR MITIGATING ABIOTIC STRESS IN PLANTS

(71) Applicant: ACADIAN SEAPLANTS LIMITED, Dartmouth (CA)

(72) Inventors: Timo Van der Zwan, Burnaby (CA); Rachel Fowler, Nova Scotia (CA); Robbie Haines, Malvern (GB); Kevin Shiell, Cornwallis (CA); Frank Prescott, Malvern (GB)

(21) Appl. No.: 18/863,601

(22) PCT Filed: May 5, 2023
(86) PCT No.: PCT/IB2023/054724

§ 371 Date: Nov. 6, 2024

(30) Foreign Application Priority Data
May 6, 2022 (GB) 2206679.9

(51) Int. Cl. A01N 37/44; A01N 65/03; A61P 21/00

(52) U.S. Cl. CPC A01N 37/44; A01N 65/03; A61P 21/00

(57) ABSTRACT

A method of preventing or mitigating the effects of abiotic stress in a plant involves administering to the plant, including a seed of the plant, or to the growth medium of the plant an effective amount of L-carnitine and a seaweed extract.

### TECHNICAL FIELD

This disclosure generally relates to the field of methods and compositions for mitigating abiotic stress in plants.

### BACKGROUND OF THE ART

Various strategies are currently used to mitigate the effects of different forms of abiotic stress on plants. Many of these are directed at physically mitigating the effect of specific stressors e.g. the application or water or shielding material to address temperature extremes or light intensity. Other approaches involve genetically engineering plants to be more resilient to stresses such as elevated salinity or temperature extremes.

These types of measures can be labour intensive and consequentially expensive, may only be useful for mitigating the effects of specific stressors and/or may only be applicable to specific plant species.

As a consequence, there remains a need for alternate methods, compounds and compositions that are effective at mitigating the impact of abiotic stress on plants, while being sustainable, cost-effective, and simple to use via current agricultural practices.

Disclosed herein, the present invention overcomes previous shortcomings in the art by providing novel compositions and methods for the treatment and mitigation of abiotic stresses in plants.

### SUMMARY OF THE INVENTION

A method of preventing or mitigating the effects of abiotic stress in a plant is provided that includes administering to the plant, a seed of the plant or to a growth medium of the plant an effective amount of a combination comprising L-carnitine and seaweed extract.

Also provided is a plant biostimulant composition having as active ingredients L-carnitine and a seaweed extract.

In one embodiment, the method comprises administering 50-800 g of L-carnitine per tonne of seed and administering 50-800 g of seaweed extract per tonne of seed.

In some embodiments, the seaweed used to produce the extract is from the class Phaeophyceae, in some embodiments from the species Ascophyllum nodosum. The seaweed extract may be produced using an alkaline hydrolysis extraction.

In some embodiments the combination consists or consists essentially of L-carnitine and seaweed extract and, optionally, water.

In some embodiments, the plant is a cereal or legume, optionally, wheat, corn or soybean.

In some embodiments, the weight ratio of the L-carnitine to the seaweed extract used in the composition or method is between 10:1 and 1:10, optionally between 2:1 to 1:2, in some embodiments, approximately 1:1. In some embodiments, the composition is an aqueous composition containing 100 to 250 g/L of seaweed extract and 100 to 250 g/L of the L-carnitine.

Also provided is a seed treatment that includes or is a composition as defined herein. In one embodiment, the L-carnitine and the seaweed extract are both in particulate form.

Also provided is a kit that includes a first package containing L-carnitine and a second package containing seaweed extract and directions for combining the L-carnitine and the seaweed extract to produce an effective amount of a plant biostimulant composition. The plant biostimulant composition is suitably a plant biostimulant composition as defined above.

In use, the method, biostimulant or seed composition may prevent or mitigate one or more of the indicia of abiotic stress identified herein.

### BRIEF DESCRIPTION OF THE DRAWINGS

FIG. 1 provides an example of improved vigour 21 days after sowing in winter wheat (cv. Marius) seed-treated with Ascophyllum nodosum extract (ANE), L-carnitine (CAR), or combinations thereof when subjected to drought stress (50% reduced irrigation) four days post-emergence, in comparison to a stressed and unstressed control according to Example 1.

FIG. 2 provides an example of improved vigour 42 days after sowing in winter wheat (cv. Marius) seed-treated with ANE, CAR or combinations thereof when subjected to drought stress (50% reduced irrigation) four days post-emergence, in comparison to a stressed and unstressed control according to Example 1.

FIG. 3 provides an example of increased foliar fresh weight 50 days after sowing in winter wheat (cv. Marius) seed-treated with ANE, CAR, or combinations thereof when subjected to drought stress (50% reduced irrigation) four days post-emergence, in comparison to a stressed and unstressed control according to Example 1.

FIG. 4 provides an example of increased root fresh weight 50 days after sowing in winter wheat (cv. Marius) seed-treated with ANE, CAR, or combinations thereof when subjected to drought stress (50% reduced irrigation) four days post-emergence, in comparison to a stressed and unstressed control according to Example 1.

FIG. 5 provides an example of increased plant height 22 days after sowing in winter wheat (cv. Marius) seed-treated with ANE, CAR, or combinations thereof when subjected to salinity stress (irrigation with 8 g/l NaCl) four days post-emergence, in comparison to a stressed and unstressed control according to Example 2.

FIG. 6 provides an example of increased plant height 35 days after sowing in winter wheat (cv. Marius) seed-treated with ANE, CAR, or combinations thereof when subjected to salinity stress (irrigation with 8 g/l NaCl) four days post-emergence, in comparison to a stressed and unstressed control according to Example 2.

FIG. 7 provides an example of increased relative chlorophyll content (SPAD) 29 days after sowing in winter wheat (cv. Marius) seed-treated with ANE, CAR, or combinations thereof when subjected to salinity stress (irrigation with 8 g/l NaCl) four days post-emergence, in comparison to a stressed and unstressed control according to Example 2.

FIG. 8 provides an example of increased plant vigour 43 days after sowing in winter wheat (cv. Marius) seed-treated with ANE, CAR, or combinations thereof when subjected to salinity stress (irrigation with 8 g/l NaCl) four days post-emergence, in comparison to a stressed and unstressed control according to Example 2.

FIG. 9 provides an example of increased fresh foliar biomass 50 days after sowing in winter wheat (cv. Marius) seed-treated with ANE, CAR, or combinations thereof when subjected to salinity stress (irrigation with 8 g/l NaCl) four days post-emergence, in comparison to a stressed and unstressed control according to Example 2.

FIG. 10 provides an example of increased plant vigour 51 days after sowing in soybean (cv. Mungo) seed-treated with ANE, CAR, or combinations thereof when subjected to drought stress (50% reduced irrigation) four days post-emergence, in comparison to a stressed and unstressed control according to Example 3.

FIG. 11 provides an example of increased foliar fresh weight 51 days after sowing in soybean (cv. Mungo) seed-treated with ANE, CAR, or combinations thereof when subjected to drought stress (50% reduced irrigation) four days post-emergence, in comparison to a stressed and unstressed control according to Example 3.

FIG. 12 provides an example of increased foliar dry weight 51 days after sowing in soybean (cv. Mungo) seed-treated with ANE, CAR, or combinations thereof when subjected to drought stress (50% reduced irrigation) four days post-emergence, in comparison to a stressed and unstressed control according to Example 3.

FIG. 13 provides an example of increased root length 10 days after sowing in corn (cv. H140354) seed-treated with ANE, CAR, or a combination thereof when subjected to drought stress (75% reduced irrigation) three days post-sowing, in comparison to a stressed and unstressed control according to Example 4.

FIG. 14 provides an example of increased photosynthetic efficiency (Phi2) 10 days after sowing in corn (cv. H140354) seed-treated with ANE, CAR, or a combination thereof when subjected to drought stress (75% reduced irrigation) three days post-sowing, in comparison to a stressed and unstressed control according to Example 4.

FIG. 15 provides an example of reduced non-photochemical quenching (NPQt, an indicator of plant stress level) 10 days after sowing in corn (cv. H140354) seed-treated with ANE, CAR, or a combination thereof when subjected to drought stress (75% reduced irrigation) three days post-sowing, in comparison to a stressed and unstressed control according to Example 4.

FIG. 16 provides an example of increased linear electron flow (LEF) of the plant photosystems 10 days after sowing in corn (cv. H140354) seed-treated with ANE, CAR, or a combination thereof when subjected to drought stress (75% reduced irrigation) three days post-sowing, in comparison to a stressed and unstressed control according to Example 4.

FIG. 17 provides an example of increased photosynthetic efficiency (Phi2) 10 days after sowing in corn (cv. H140354) seed-treated with ANE, CAR, or a combination thereof when subjected to salinity stress (10 ml of 58 g/l NaCl) three days post-sowing, in comparison to a stressed and unstressed control according to Example 5.

FIG. 18 provides an example of increased linear electron flow (LEF) of the plant photosystems 10 days after sowing in corn (cv. H140354) seed-treated with ANE, CAR, or a combination thereof when subjected to salinity stress (10 ml of 58 g/l NaCl) three days post-sowing, in comparison to a stressed and unstressed control according to Example 5.

FIG. 19 provides an example of reduced non-photochemical quenching (NPQt, an indicator of plant stress levels) 10 days after sowing in corn (cv. H140354) seed-treated with ANE, CAR, or a combination thereof when subjected to salinity stress (10 ml of 58 g/l NaCl) three days post-sowing, in comparison to a stressed and unstressed control according to Example 5.

FIG. 20 provides an example of reduced non-photochemical quenching (NPQt, an indicator of plant stress levels) 10 days after sowing in wheat (cv. Skyfall, SEED-0035) seed-treated with ANE, CAR, or a combination thereof when subjected to cold stress in comparison to a stressed and unstressed control according to Example 6. Center lines show the medians; box limits indicate the 25th and 75th percentiles; whiskers extend to data points that are less than 1.5 times the interquartile range away from the first/third quartile.

FIG. 21 provides an example of reduced non-photochemical quenching (NPQt, an indicator of plant stress levels) 17 days after sowing in soybean (cv. Marula, SEED-003) seed-treated with ANE, CAR, or a combination thereof when subjected to salinity stress in comparison to a stressed and unstressed control according to Example 7.1. Center lines show the medians; box limits indicate the 25th and 75th percentiles; whiskers extend to data points that are less than 1.5 times the interquartile range away from the first/third quartile.

FIG. 22 provides an example of increased shoot weight 17 days after sowing in soybean (cv. Marula, SEED-003) seed-treated with ANE, CAR, or a combination thereof when subjected to salinity stress in comparison to a stressed and unstressed control according to Example 7.2. Center lines show the medians; box limits indicate the 25th and 75th percentiles; whiskers extend to data points that are less than 1.5 times the interquartile range away from the first/third quartile.

### DETAILED DESCRIPTION OF THE INVENTION

As used herein, “abiotic stress” refers to the negative impact of non-living factors on plants; these factors can include, but are not necessarily limited to: extremes of heat and cold, drought or flooding water stress, high or low light intensity, elevated salinity, and soil toxicity. Thus, the consequences of abiotic stress are typically those consequences which negatively impact crop yield and quality.

As used herein, “plants” may refer to any organism of the kingdom Plantae. In preferred embodiments, the plant is a terrestrial plant. In some embodiments, a crop. Unless context dictates otherwise, it will be understood that administering a compound or composition as described herein to a plant comprises administering the compound or composition to any part of the plant and at any stage of growth, e.g. to a seed, roots or leaves of a plant.

As used herein, a “biostimulant” refers to a product composed of substances, micro-organisms, and/or other materials able to stimulate nutrition processes independently of its nutrient content, either in the plant or its surrounding growing environment, that improve the plant's nutrition use efficiency, tolerance to abiotic stress, and/or the crop quality.

As used herein, “mitigating the effects of abiotic stress” refers to reducing, reversing or preventing one or more indicia of abiotic stress better than a control plant or part thereof (i.e., a plant or part thereof that has been exposed to the same abiotic stress but has not been contacted with the compositions of the present invention).

Methods of identifying and measuring indicia of abiotic stress in plants are known to those of skill in the art and can include visual assessments of plant vitality, such as a reduction in the number or size of plants or parts thereof, a reduction in seed germination or emergence, or a reduction in seedling growth rates or vigour; gravimetric assessment of biomass yield, such as fresh or dry weights of shoots or roots; optical scanner-based assessments of plant parts, such as scanning of leaves or root systems and algorithmic determinations of leaf area or root lengths; physiological or biochemical assessments, such as cell membrane stability or relative leaf water content; and photosynthetic assessments of plant stress levels using reflectance or spectroscopy-based methods, such as determination of photosynthetic efficiency, linear electron flow, non-photochemical quenching, and relative chlorophyll levels.

As used herein, an “effective amount” is an amount sufficient to mitigate the effects of abiotic stress, which may be evaluated using one or more of the indicia discussed above. The effective amount will vary with type and condition of the plant, as well as the nature and duration of the abiotic stressor (or expected abiotic stressor in the case of a preventative treatment).

As used herein, “growth medium” is used generically to refer to the substrate on which a plant is anchored or grown, and may include soil or man-made mediums, including e.g. mediums suitable for hydroponic applications, such as rockwool or vermiculite.

As used herein, “L-carnitine” (p-hydroxy-y-N-trimethylaminobutyric acid) is a derivative of the amino acid lysine. It was first isolated from meat in 1905 and has a critical role in the oxidation of long chain fatty acids for energy production in animal tissues. Commercial uses of L-carnitine include as a dietary supplement and as a foam booster in some cosmetic products.

Naturally occurring amino acids which include carnitine, phenylalanine, lysine, etc. can be obtained by extraction, fermentation, enzymatic reaction and synthesis. Methods for the synthetic manufacture of L-carnitine are known to those of skill in the art, and L-carnitine is commercially available from a variety of sources.

Marine algae have been used as agricultural inputs for hundreds of years. It was first used as a soil amendment where the seaweed was incorporated into the growing media and broken down by soil microbes More recently it has served as a raw material for the production of “seaweed extract” which is used as a biostimulant. Species such as Ascophyllum nodosum, Durvillaea potatorum, Ecklonia Maxima, Ulva Lactuca, and Gelidium robustum have been used in the manufacture of plant biostimulants and, in some embodiments, may be used in the methods and compositions provided herein.

As used herein, “seaweed extract” refers to products obtained from marine algae, preferably red, brown or green macroalgae, through a process that extracts and concentrates bio-stimulating compounds. Methods of obtaining extracts from seaweed are known to those of skill in the art, see e.g. E L Boukhari MEM, Barakate M, Bouhia Y, Lyamlouli K. Trends in Seaweed Extract Based Biostimulants: Manufacturing Process and Beneficial Effect on Soil-Plant Systems. Plants 2020, 9, 359. doi: 10.3390/plants9030359. Further, in some embodiments, the seaweed extract may be prepared from a combination of seaweed species.

Seaweed processing/extraction can be made using solvents, acids, bases, enzymes or mechanical means eventually in any combination. Preferably, the processing/extraction is made by contacting the seaweed with an aqueous solution comprising an alkaline extraction agent.

For the purpose of the present invention, the base is preferably an inorganic base selected from: NaOH, KOH, Na₂CO₃, K₂CO₃, or any combination thereof. The alkaline solvent concentration ranges from 1% to 10% w/w, more specifically 2% to 5% w/w.

Preferably, the temperature of the extraction step ranges preferably between 20° C. and 100° C. and the extraction time ranges from 30 minutes to 18 hours at pressures ranging from 1 to 6 Bar.

The extraction step may be followed by a further step of separating/removing the non-solubilized components when it is desirable using only the extract in the formulation. The removing/separating step is preferably performed by decantation, filtration or centrifugation. Alternatively, a suspension comprising both the extracted components and the non-extracted components can be used.

According to a preferred embodiment, the seaweed extract used in the invention is prepared from the brown marine algae Ascophyllum nodosum. The resulting extract may be in the form of a soluble liquid, a liquid concentrate, or a water-soluble powder.

Provided herein is a plant biostimulant composition comprising an effective amount of at least one amino-acid betaine and a seaweed extract and, in particular, L-carnitine and preferably a seaweed extract from the class Phaeophyceae, more preferably from the species Ascophyllum nodosum. In some embodiments, the active ingredients of the biostimulant composition are limited to L-carnitine, and the seaweed extract, preferably Ascophyllum nodosum. In some embodiments, the biostimulant composition, consists or consists essentially of L-carnitine, and the seaweed extract, preferably Ascophyllum nodosum and, optionally, water. In this context, “active ingredient” can be understood as ingredients having observable biostimulant effect, i.e. of preventing or mitigating the effects of abiotic stress, which may be identified or measured based on one or more indicia identified herein, as distinct from e.g. excipients that either by their nature or as a result of the trace amounts present in the combination do not have such observable effect.

The efficacy of the combination of ANE and L-carnitine in mitigating the effects of abiotic stress is evident from the Examples. In view of the evidence of efficacy, additional experiments were performed to evaluate synergistic effect of ANE and L-carnitine in mitigating abiotic stress. As detailed in the Examples, there was evidence supporting synergistic effect when plants were subject to different forms of abiotic stress, including cold and salinity.

In one embodiment, the biostimulant composition may be in the form of a powder containing the amino-acid betaine, preferably L-carnitine, and a seaweed extract powder. The composition may be dispersed in a liquid medium to facilitate application.

In some embodiments, the weight ratio of a combination of L-carnitine to the seaweed extract in the composition or used in the method provided is between 10:1 and 1:10, in some embodiments between 2:1 to 1:2. In some embodiments, approximately equals amounts by weight of L-carnitine and the seaweed extract are used.

In some embodiments, the biostimulant composition containing L-carnitine and Ascophyllum nodosum extract may be in the form of a liquid, which may be particularly suitable for foliar application or root drenches.

In some embodiment, the compositions provided herein may be formulated as an aqueous concentrate containing seaweed extract, preferably Ascophyllum nodosum extract, at a range of 100 to 250 g/l in combination with L-carnitine at 100 to 250 g/l.

In some embodiments, the compositions as described herein may be used as a seed treatment. Seed treatment formulations and technologies are commercially available and methods of applying seed treatments are known to those of skill in the art and can include direct application of a seed treatment to the seed using commercially available seed treating equipment.

According to the scope of conditions, an effective amount typically ranges from 50-800 g L-carnitine per tonne of seed and 50-800 g seaweed extract, preferably Ascophyllum nodosum extract, per tonne of seed for seed treatment.

In a preferred embodiment, the composition described herein for use as a seed treatment may be formulated as an aqueous concentrate containing Ascophyllum nodosum extract at a range of 100 to 250 g/l in combination with L-carnitine at 100 to 250 g/l.

The compositions provided herein may also contain suitable formulation excipients known to persons of skill in the art, and which are commercially available. In some embodiments, formulation excipients are suitably selected from, but not exclusively, surfactants, wetting and dispersing agents, stickers/binders, antifreeze, antifoams, preservatives, rheological aids and, if appropriate, colourants.

The treatments described herein may also be preventative, e.g. methods and compositions provided herein may be provided as a seed treatment prior to an expected stressor event, such as a cold snap or heat wave to mitigate the effects of the stressor.

While in its broadest embodiments, the methods, compounds and compositions described herein may be used in the cultivation of any plant, in some embodiments, they may be used in the cultivation of commercially important plants, which can include, without being limited to, cereals (including maize, wheat, alfalfa, barley, rye, oat), vegetables (including pepper, tomato, lettuce, carrots, potatoes), and other important crops, such as cotton, rice, soybean, canola, oilseed rape.

All documents referenced herein are incorporated by reference, however, it should be appreciated that any patent, publication, or other disclosure material, in whole or in part, that is incorporated by reference herein is incorporated only to the extent that the incorporated material does not conflict with definitions, statements, or other disclosure material set forth in this disclosure. As such, and to the extent necessary, the disclosure as explicitly set forth herein supersedes any conflicting material incorporated herein by reference.

It will be understood that numerous modifications thereto will appear to those skilled in the art. Accordingly, the above description and accompanying drawings should be taken as illustrative of the invention and not in a limiting sense. It will further be understood that it is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

### EXAMPLES

### Example 1: Wheat Drought Stress Greenhouse Trial

Winter wheat seed (cv. Marius) was treated at a total application volume of 10 ml/kg with water (unstressed control and stressed control treatment groups), or an aqueous solution of Ascophyllum nodosum extract (ANE) at 1 ml/kg, or an aqueous solution of L-carnitine hydrochloride (CAR) at 0.3 g/kg, or combinations of Ascophyllum nodosum extract at 1 ml/kg with either 0.1, 0.2, 0.3, or 0.4 g/kg L-carnitine hydrochloride. Treated wheat seed was sown in soil in pots with 10 biological replicates per treatment group in a randomized complete block design. Four days after full emergence, drought stress was imposed on all but the unstressed control treatment group through a 50% reduced irrigation regime relative to the full irrigation received by the unstressed control group.

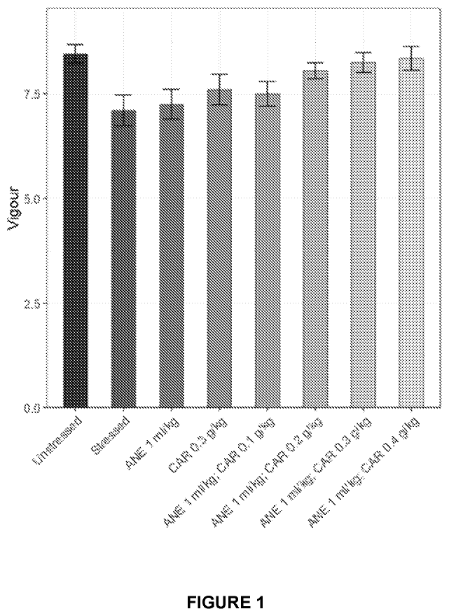

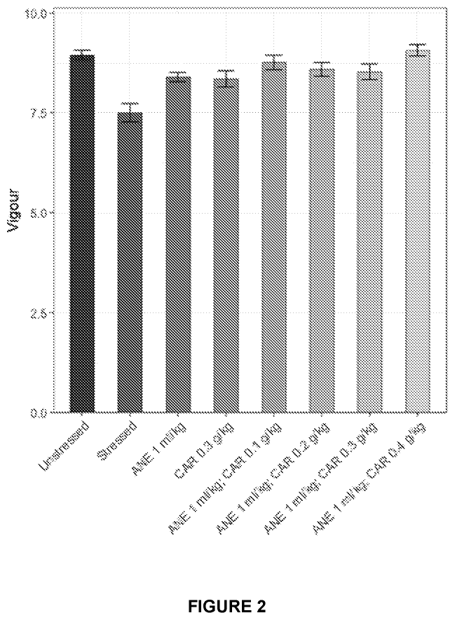

As evidenced in FIG. 1 and Table 1, plant vigour (assessed on a 0-10 scale with 0 being plants having no foliage and 10 representing full vigour, being standard foliage vigour of the crop in normal conditions) was increased by up to 17.6% compared to the stressed control in the plants seed-treated with ANE at 1 ml/kg and CAR at 0.4 g/kg at 21 days after sowing. By 42 days after sowing, this difference had increased to 20.8% compared to the stressed control, as shown in FIG. 2 and Table 2.

TABLE 1

| Treatment group | Mean | Difference (%) |
|---|---|---|
| Unstressed | 8.45 | 19.0 |
| Stressed | 7.10 | 0.0 |
| ANE 1 ml/kg | 7.25 | 2.1 |
| CAR 0.3 g/kg | 7.60 | 7.0 |
| ANE 1 ml/kg; CAR 0.1 g/kg | 7.50 | 5.6 |
| ANE 1 ml/kg; CAR 0.2 g/kg | 8.05 | 13.4 |
| ANE 1 ml/kg; CAR 0.3 g/kg | 8.25 | 16.2 |
| ANE 1 ml/kg; CAR 0.4 g/kg | 8.35 | 17.6 |

TABLE 2

| Treatment group | Mean | Difference (%) |
|---|---|---|
| Unstressed | 8.95 | 19.3 |
| Stressed | 7.50 | 0.0 |
| ANE 1 ml/kg | 8.40 | 12.0 |
| CAR 0.3 g/kg | 8.35 | 11.3 |
| ANE 1 ml/kg; CAR 0.1 g/kg | 8.76 | 16.9 |
| ANE 1 ml/kg; CAR 0.2 g/kg | 8.59 | 14.5 |
| ANE 1 ml/kg; CAR 0.3 g/kg | 8.53 | 13.7 |
| ANE 1 ml/kg; CAR 0.4 g/kg | 9.06 | 20.8 |

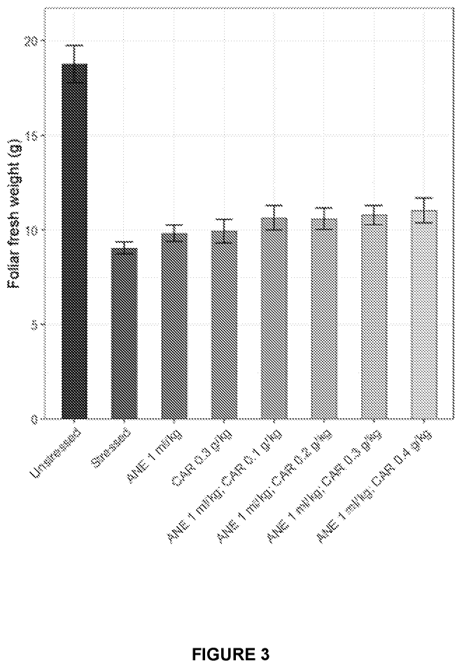

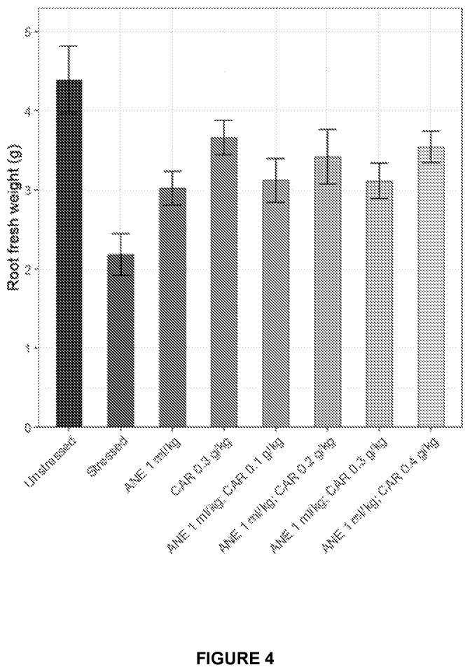

The improved plant vigour afforded by seed treatment with the combination of ANE and CAR translated to increased biomass yield assessed 50 days after sowing, with an up to 22.1% increase in foliar fresh weight (FIG. 3, Table 3) and a 62% increase in fresh root weight in the plants seed-treated with ANE at 1 ml/kg and CAR at 0.4 g/kg (FIG. 4, Table 4).

TABLE 3

| Treatment group | Mean | Difference (%) |
|---|---|---|
| Unstressed | 18.77 | 107.6 |
| Stressed | 9.04 | 0.0 |
| ANE 1 ml/kg | 9.83 | 8.7 |
| CAR 0.3 g/kg | 9.94 | 9.9 |
| ANE 1 ml/kg; CAR 0.1 g/kg | 10.64 | 17.6 |
| ANE 1 ml/kg; CAR 0.2 g/kg | 10.59 | 17.1 |
| ANE 1 ml/kg; CAR 0.3 g/kg | 10.78 | 19.2 |
| ANE 1 ml/kg; CAR 0.4 g/kg | 11.04 | 22.1 |

TABLE 4

| Treatment group | Mean | Difference (%) |
|---|---|---|
| Unstressed | 4.39 | 100.9 |
| Stressed | 2.18 | 0.0 |
| ANE 1 ml/kg | 3.02 | 38.2 |
| CAR 0.3 g/kg | 3.66 | 67.5 |
| ANE 1 ml/kg; CAR 0.1 g/kg | 3.12 | 42.8 |
| ANE 1 ml/kg; CAR 0.2 g/kg | 3.42 | 56.5 |
| ANE 1 ml/kg; CAR 0.3 g/kg | 3.11 | 42.3 |
| ANE 1 ml/kg; CAR 0.4 g/kg | 3.54 | 62.0 |

### Example 2: Wheat Salinity Stress Greenhouse Trial

Winter wheat seed (cv. Marius) was treated at a total application volume of 10 ml/kg with water (unstressed control and stressed control treatment groups), or an aqueous solution of Ascophyllum nodosum extract (ANE) at 1 ml/kg, or an aqueous solution of L-carnitine hydrochloride (CAR) at 0.3 g/kg, or combinations of Ascophyllum nodosum extract at 1 ml/kg with either 0.1, 0.2, 0.3, or 0.4 g/kg L-carnitine hydrochloride. Treated wheat seed was sown in pots containing soil with 10 biological replicates per treatment group in a randomized complete block design. Four days after full emergence, salinity stress was imposed on all but the unstressed control treatment group through irrigation with saline water (8 g/l NaCl) for the remainder of the trial. The unstressed control treatment group was irrigated with non-saline water.

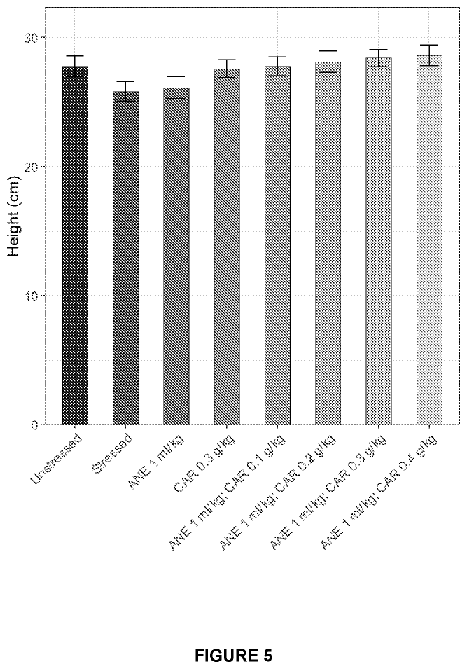

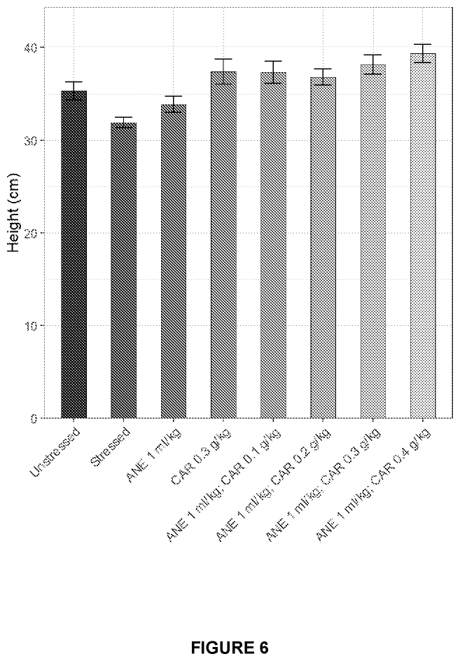

As evidenced in FIG. 5 and Table 5, plant height was increased by up to 10.9% compared to the stressed control in the plants seed-treated with ANE at 1 ml/kg and CAR at 0.4 g/kg at 22 days after sowing. By 35 days after sowing, the height increase in this treatment group had increased to 23.3% in comparison to the stressed control (FIG. 6, Table 6).

TABLE 5

| Treatment group | Mean | Difference (%) |
|---|---|---|
| Unstressed | 27.75 | 7.6 |
| Stressed | 25.80 | 0.0 |
| ANE 1 ml/kg | 26.10 | 1.2 |
| CAR 0.3 g/kg | 27.55 | 6.8 |
| ANE 1 ml/kg; CAR 0.1 g/kg | 27.75 | 7.6 |
| ANE 1 ml/kg; CAR 0.2 g/kg | 28.10 | 8.9 |
| ANE 1 ml/kg; CAR 0.3 g/kg | 28.40 | 10.1 |
| ANE 1 ml/kg; CAR 0.4 g/kg | 28.60 | 10.9 |

TABLE 6

| Treatment group | Mean | Difference (%) |
|---|---|---|
| Unstressed | 35.30 | 10.7 |
| Stressed | 31.90 | 0.0 |
| ANE 1 ml/kg | 33.85 | 6.1 |
| CAR 0.3 g/kg | 37.39 | 17.2 |
| ANE 1 ml/kg; CAR 0.1 g/kg | 37.33 | 17.0 |
| ANE 1 ml/kg; CAR 0.2 g/kg | 36.80 | 15.4 |
| ANE 1 ml/kg; CAR 0.3 g/kg | 38.16 | 19.6 |
| ANE 1 ml/kg; CAR 0.4 g/kg | 39.33 | 23.3 |

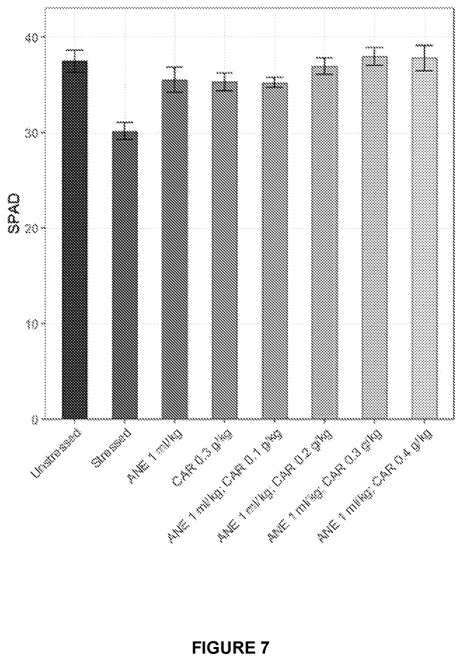

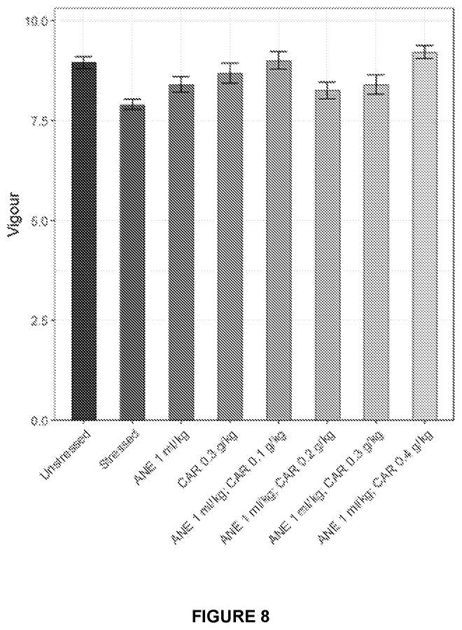

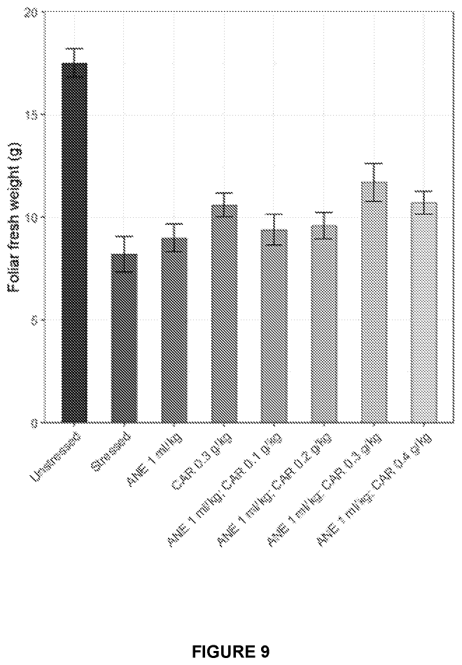

At 29 days after sowing, the plant relative chlorophyll content (SPAD) showed a 25.5% increase in the same treatment group in comparison to the stressed control (FIG. 7, Table 7). [A similar mitigating effect on photosynthetic rate and/or capacity as evidenced by SPAD was seen in corn plants subject to cold stress under a protocol akin to Examples 6 and 7, below.] The increased stress tolerance of the plants was likewise reflected in the plant vigour assessment at 43 days after seeding, with an up to 16.6% increase over the stressed control (FIG. 8, Table 8). By 50 days after seeding, an up to 42.7% increase in fresh foliar biomass was observed in the plants seed-treated with ANE at 1 ml/kg and CAR at 0.3 g/kg, demonstrating strong induction of long-lasting salinity stress tolerance from the combined treatment of ANE and CAR (FIG. 9, Table 9).

TABLE 7

| Treatment group | Mean | Difference (%) |
|---|---|---|
| Unstressed | 37.46 | 24.3 |
| Stressed | 30.14 | 0.0 |
| ANE 1 ml/kg | 35.52 | 17.8 |
| CAR 0.3 g/kg | 35.30 | 17.1 |
| ANE 1 ml/kg; CAR 0.1 g/kg | 35.24 | 16.9 |
| ANE 1 ml/kg; CAR 0.2 g/kg | 36.94 | 22.6 |
| ANE 1 ml/kg; CAR 0.3 g/kg | 37.98 | 26.0 |
| ANE 1 ml/kg; CAR 0.4 g/kg | 37.81 | 25.5 |

TABLE 8

| Treatment group | Mean | Difference (%) |
|---|---|---|
| Unstressed | 8.95 | 13.3 |
| Stressed | 7.90 | 0.0 |
| ANE 1 ml/kg | 8.40 | 6.3 |
| CAR 0.3 g/kg | 8.68 | 9.9 |
| ANE 1 ml/kg; CAR 0.1 g/kg | 9.00 | 13.9 |
| ANE 1 ml/kg; CAR 0.2 g/kg | 8.25 | 4.4 |
| ANE 1 ml/kg; CAR 0.3 g/kg | 8.40 | 6.3 |
| ANE 1 ml/kg; CAR 0.4 g/kg | 9.21 | 16.6 |

TABLE 9

| Treatment group | Mean | Difference (%) |
|---|---|---|
| Unstressed | 17.5 | 113.4 |
| Stressed | 8.2 | 0.0 |
| ANE 1 ml/kg | 9.0 | 9.8 |
| CAR 0.3 g/kg | 10.6 | 29.3 |
| ANE 1 ml/kg; CAR 0.1 g/kg | 9.4 | 14.6 |
| ANE 1 ml/kg; CAR 0.2 g/kg | 9.6 | 17.1 |
| ANE 1 ml/kg; CAR 0.3 g/kg | 11.7 | 42.7 |
| ANE 1 ml/kg; CAR 0.4 g/kg | 10.7 | 30.5 |

### Example 3: Soybean Drought Stress Greenhouse Trial

Soybean seed (cv. Mungo) was treated at a total application volume of 10 ml/kg with water (unstressed control and stressed control treatment groups), or an aqueous solution of Ascophyllum nodosum extract (ANE) at 1 ml/kg, or an aqueous solution of L-carnitine hydrochloride (CAR) at 0.3 g/kg, or combinations of Ascophyllum nodosum extract at 1 ml/kg with either 0.1, 0.2, 0.3, or 0.4 g/kg L-carnitine hydrochloride. Treated soybean seed was sown in pots containing soil with 10 biological replicates per treatment group in a randomized complete block design. Four days after full emergence, drought stress was imposed on all but the unstressed control treatment group through a 50% reduced irrigation regime relative to the full irrigation received by the unstressed control group.

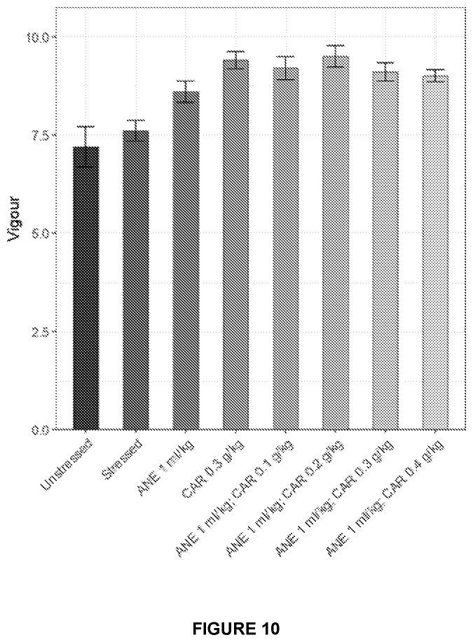

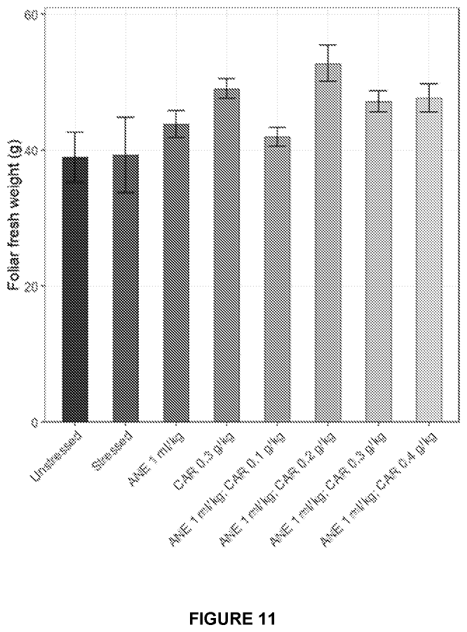

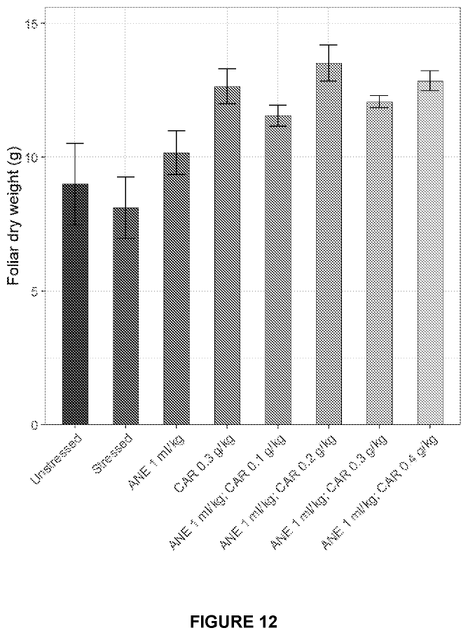

As evidenced in FIG. 10 and Table 10, plant vigour was increased by up to 25% compared to the stressed control in the plants seed-treated with ANE at 1 ml/kg and CAR at 0.2 g/kg at 51 days after sowing. Correspondingly, the foliar fresh weight of this treatment group showed a 34.4% increase compared to the stressed control (FIG. 11, Table 11), and a 66.7% increase in foliar dry weight (FIG. 12, Table 12). This demonstrates the clear mitigation of the yield-reducing effects of the induced drought stress afforded by the combined seed treatment of ANE and CAR. A similar protective effect on shoot fresh weight at 17 days post sowing was observed for soybean plants germinated from seed treated with a combination of 0.36 g ANE and 0.30 g L-carnitine subject to drought stress under a separate protocol.

TABLE 10

| Treatment group | Mean | Difference (%) |
|---|---|---|
| Unstressed | 7.2 | −5.3 |
| Stressed | 7.6 | 0.0 |
| ANE 1 ml/kg | 8.6 | 13.2 |
| CAR 0.3 g/kg | 9.4 | 23.7 |
| ANE 1 ml/kg; CAR 0.1 g/kg | 9.2 | 21.1 |
| ANE 1 ml/kg; CAR 0.2 g/kg | 9.5 | 25.0 |
| ANE 1 ml/kg; CAR 0.3 g/kg | 9.1 | 19.7 |
| ANE 1 ml/kg; CAR 0.4 g/kg | 9.0 | 18.4 |

TABLE 11

| Treatment group | Mean | Difference (%) |
|---|---|---|
| Unstressed | 39.0 | −0.8 |
| Stressed | 39.3 | 0.0 |
| ANE 1 ml/kg | 43.9 | 11.7 |
| CAR 0.3 g/kg | 49.1 | 24.9 |
| ANE 1 ml/kg; CAR 0.1 g/kg | 42.0 | 6.9 |
| ANE 1 ml/kg; CAR 0.2 g/kg | 52.8 | 34.4 |
| ANE 1 ml/kg; CAR 0.3 g/kg | 47.2 | 20.1 |
| ANE 1 ml/kg; CAR 0.4 g/kg | 47.7 | 21.4 |

TABLE 12

| Treatment group | Mean | Difference (%) |
|---|---|---|
| Unstressed | 8.99 | 10.9 |
| Stressed | 8.11 | 0.0 |
| ANE 1 ml/kg | 10.17 | 25.4 |
| CAR 0.3 g/kg | 12.64 | 55.9 |
| ANE 1 ml/kg; CAR 0.1 g/kg | 11.54 | 42.4 |
| ANE 1 ml/kg; CAR 0.2 g/kg | 13.52 | 66.7 |
| ANE 1 ml/kg; CAR 0.3 g/kg | 12.07 | 48.8 |
| ANE 1 ml/kg; CAR 0.4 g/kg | 12.85 | 58.4 |

### Example 4: Corn Drought Stress Controlled Environment Room Trial

Corn seed (cv. H140354) was treated at a total application volume of 10 ml/kg with water (unstressed control and stressed control treatment groups), or an aqueous solution of Ascophyllum nodosum extract (ANE) at 2 ml/kg, or an aqueous solution of L-carnitine hydrochloride (CAR) at 0.3 g/kg, or a combinations of Ascophyllum nodosum extract at 2 ml/kg and 0.3 g/kg L-carnitine hydrochloride. Treated corn seed was sown in field soil inside cell packs set in greenhouse trays with 16 biological replicates per treatment group in a randomized complete block design. Three days after seeding, drought stress was imposed on all but the unstressed control treatment group through a 75% reduced irrigation regime relative to the full irrigation received by the unstressed control group, and assessments were made ten days after seeding.

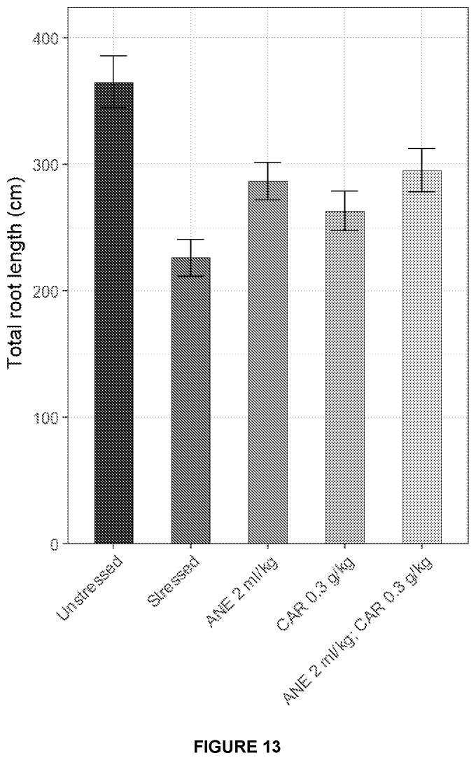

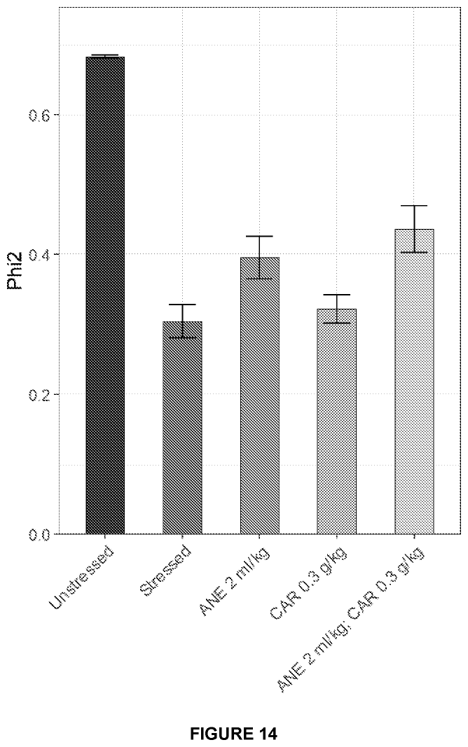

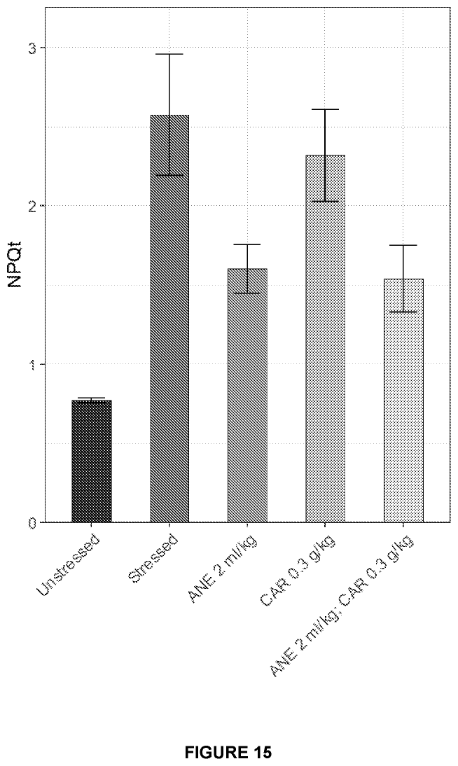

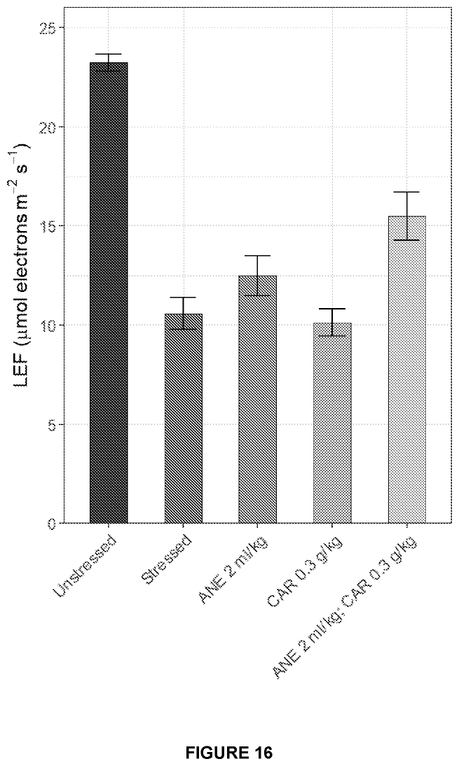

As evidenced in FIG. 13 and Table 13, seed treatment with ANE at 2 ml/kg and CAR at 0.3 g/kg resulted in a 30.6% increase in corn root length in comparison to the stressed control at 10 days after sowing. The combined seed treatment of ANE and CAR furthermore resulted in a 43.2% increased level of photosynthetic efficiency (photosystem II efficiency, Phi2) (FIG. 14, Table 14), a 40.2% reduction in non-photochemical quenching (NPQt, which is the amount of incoming light that is regulated away from photosynthetic processes in order to reduce damage to the plant, and is an indicator of plant stress level) (FIG. 15, Table 15), and a 46.2% increase in linear electron flow (LEF) of the plant photosystems (FIG. 16, Table 16). The latter measurement in particular demonstrated a surprising and unexpected synergistic effect, where ANE at 2 ml/kg resulted in an 18% increase in LEF, CAR at 0.3 g/kg resulted in a 4.3% decrease in LEF, while their combined treatment resulted in a 46.2% increase. These results demonstrate an increased capacity to conduct photosynthesis and maintain root growth in corn plants exposed to drought stress, afforded by the combined seed treatment with ANE and CAR.

TABLE 13

| Treatment group | Mean | Difference (%) |
|---|---|---|
| Unstressed | 365.3 | 61.4 |
| Stressed | 226.3 | 0.0 |
| ANE 2 ml/kg | 286.8 | 26.8 |
| CAR 0.3 g/kg | 263.3 | 16.4 |
| ANE 2 ml/kg; CAR 0.3 g/kg | 295.4 | 30.6 |

TABLE 14

| Treatment group | Mean | Difference (%) |
|---|---|---|
| Unstressed | 0.68 | 124.0 |
| Stressed | 0.30 | 0.0 |
| ANE 2 ml/kg | 0.40 | 29.8 |
| CAR 0.3 g/kg | 0.32 | 5.7 |
| ANE 2 ml/kg; CAR 0.3 g/kg | 0.44 | 43.2 |

TABLE 15

| Treatment group | Mean | Difference (%) |
|---|---|---|
| Unstressed | 0.77 | −70.0 |
| Stressed | 2.57 | 0.0 |
| ANE 2 ml/kg | 1.60 | −37.8 |
| CAR 0.3 g/kg | 2.32 | −10.0 |
| ANE 2 ml/kg: CAR 0.3 g/kg | 1.54 | −40.2 |

TABLE 16

| Treatment group | Mean | Difference (%) |
|---|---|---|
| Unstressed | 23.24 | 119.4 |
| Stressed | 10.59 | 0.0 |
| ANE 2 ml/kg | 12.50 | 18.0 |
| CAR 0.3 g/kg | 10.14 | −4.3 |
| ANE 2 ml/kg; CAR 0.3 g/kg | 15.49 | 46.2 |

### Example 5: Corn Salinity Stress Controlled Environment Room Trial

Corn seed (cv. H140354) was treated at a total application volume of 10 ml/kg with water (unstressed control and stressed control treatment groups), or combinations of Ascophyllum nodosum extract (ANE) at 2 ml/kg with either 0.2, 0.3, or 0.4 g/kg L-carnitine hydrochloride (CAR). Treated corn seed was sown in field soil inside cell packs set in greenhouse trays with 16 biological replicates per treatment group in a randomized complete block design. Four days after seeding, salinity stress was imposed on all but the unstressed control treatment group through irrigation with saline water (10 ml of 58 g/l NaCl). The unstressed control treatment group was irr1igated with non-saline water. Assessments were made eleven days after seeding.

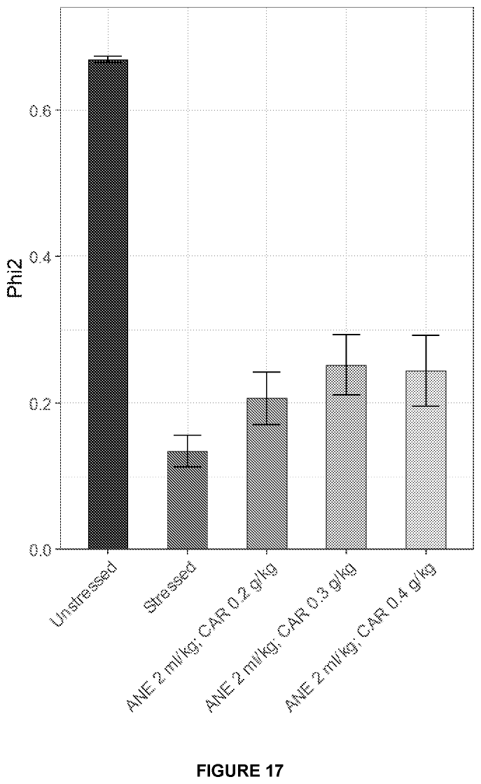

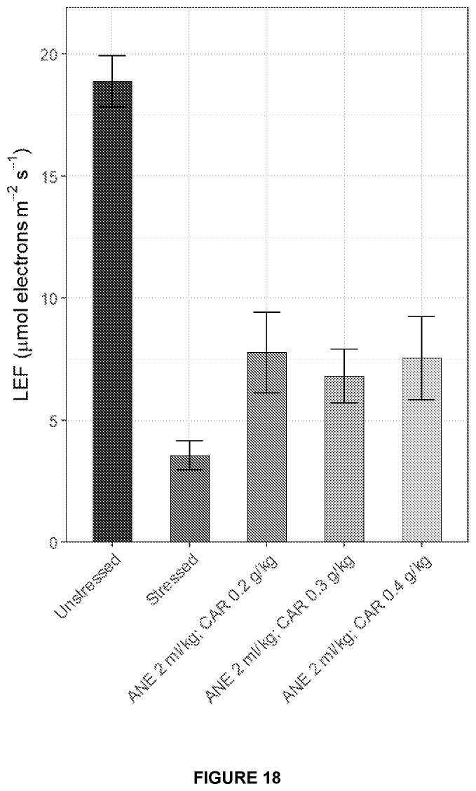

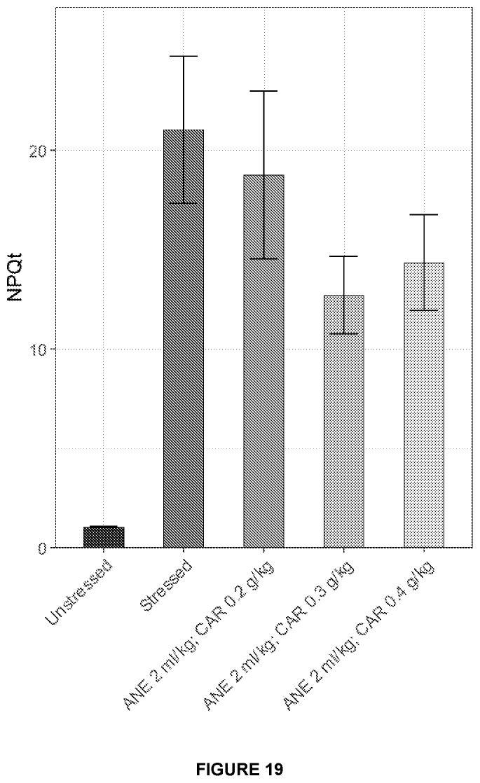

As evidenced in FIG. 17 and Table 17, the combined seed treatment with ANE 2 ml/kg and CAR 0.3 g/kg resulted in an 87.8% increased level of photosynthetic efficiency (photosystem II efficiency, Phi2) in the salt-stressed corn plants. An up to 118.5% increase in linear electron flow (LEF) of the plant photosystems (FIG. 18, Table 18) was furthermore observed, as well as an up to 39.5% reduction in non-photochemical quenching (NPQt, an indicator of plant stress level) (FIG. 19, Table 19).

TABLE 17

| Treatment group | Mean | Difference (%) |
|---|---|---|
| Unstressed | 0.67 | 398.4 |
| Stressed | 0.13 | 0.0 |
| ANE 2 ml/kg; CAR 0.2 g/kg | 0.21 | 53.8 |
| ANE 2 ml/kg; CAR 0.3 g/kg | 0.25 | 87.8 |
| ANE 2 ml/kg; CAR 0.4 g/kg | 0.24 | 81.9 |

TABLE 18

| Treatment group | Mean | Difference (%) |
|---|---|---|
| Unstressed | 18.89 | 431.1 |
| Stressed | 3.56 | 0.0 |
| ANE 2 ml/kg: CAR 0.2 g/kg | 7.77 | 118.5 |
| ANE 2 ml/kg; CAR 0.3 g/kg | 6.81 | 91.5 |
| ANE 2 ml/kg; CAR 0.4 g/kg | 7.54 | 112.2 |

TABLE 19

| Treatment group | Mean | Difference % |
|---|---|---|
| Unstressed | 1.06 | −95.0 |
| Stressed | 21.05 | 0.0 |
| ANE 2 ml/kg; CAR 0.2 g/kg | 18.77 | −10.8 |
| ANE 2 ml/kg; CAR 0.3 g/kg | 12.72 | −39.5 |
| ANE 2 ml/kg; CAR 0.4 g/kg | 14.36 | −31.8 |

### Example 6: Wheat Subject to Cold Stress

An investigation was performed to assess the effect of seed treatment on mitigating the effects of a further form of abiotic stress, namely cold stress.

Seed Treatment solutions were prepared per Table 20:

| | Amount | Amount of | Amount |
|---|---|---|---|
| Treatment group | of ANE | Carnitine | of water |
| Unstressed | | | 10 mL |
| Stressed | | | 10 mL |
| 0.36 g ANE | 2 mL | | 8 mL |
| 0.30 g Carnitine | | 2 mL | 8 mL |
| 0.36 g ANE + 0.30 g Carnitine | 2 mL | 2 mL | 6 mL |

0.1 mL of treatment solution was pipetted into a 500-mL glass beaker around the edges and 10 g of wheat seed (cultivar Skyfall, ref. SEED-0035) was added. The beaker was swirled by hand for 1 min to coat the seeds, which were then swirled intermittently until dry, if needed.

The trial was set up according to the following protocol:
1. 32 8-0-6 cell packs were labelled with coloured stickers to denote five treatments with 4 cell packs for non-stress controls and 7 for the remaining 4 treatments.
2. In a container 6 L of stored field soil and 600 mL water were mixed thoroughly.
3. If soil was too dry, an additional 300 mL water, or an amount as appropriate, was added.
4. Each cell pack was filled with pre-moistened soil; packed down once by tapping it on a counter; topped up and levelled off. Filled cell packs were placed in a quad thick flat tray in a randomized block design.
5. Steps 3 to 4 were repeated, in random order across treatment groups and replicates.
6. Once all cell packs were filled, a stamp was used to create even depth holes in each cell pack. Three seeds per cell were planted at 1″ and then covered with soil.
7. Each tray was watered with 750 mL by removing one cell pack and pouring water into the bottom of the tray. 7″ plastic dome was placed on each tray, which were grown under the following conditions: 25/20° C. day/night, 16/8 h, 70% humidity, 400 μmol m-2 s-1 PAR.

Unstressed controls were watered as needed throughout the trial. Unstressed controls were separated from the rest of the trial. Measurements were taken 10 days after sowing.

Plants were exposed to cold stress on growth chamber (conviron) for 4 days and measuments were taken 10 after sowing. Environmental conditions: 13/8° C. day/night, 16/8 h photoperiod, 60% humidity, 600 ppm and 400 μmol m⁻² s⁻¹ PAR.

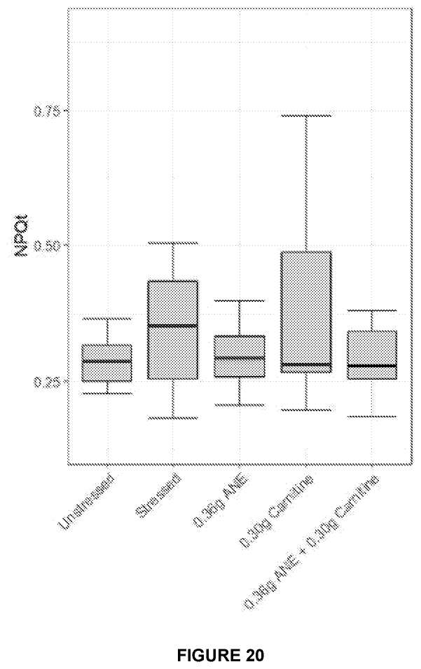

An estimate of non-photochemical quenching (NPQt), which is the amount of incoming light that is regulated away from photosynthetic processes in order to reduce damage to the plant was obtained using the MultispeQ (B-4003-AA) from PhotosynQ, according to manufaturer's directions. For the photosynthesis measuments a total of 12-14 plants per treatment were measured. Results are shown in Table 21 and FIG. 20. Parametric analysis of variance (ANOVA) was done using the aov function in the ggpubr package, and multiple comparison testing was done with the Tukey test (alpha=0.1) using the TukeyHSD function in the stats package in R.

TABLE 21

| Treatment group | Mean | Difference (%) |
|---|---|---|
| Unstressed | 0.30 | −26.6 |
| Stressed | 0.42 | 0.0 |
| 0.36 g ANE | 0.30 | −28.5 |
| 0.30 g Carnitine | 0.40 | −4.8 |
| 0.36 g ANE + 0.30 g Carnitine | 0.37 | −11.2 |

To supplement the observed evidence of efficacy, a synergy assessment was made. Synergy was calculated using two different methodologies, both of which find support in the literature: 1) Equation Ill from Colby, S. R. 1967. Calculating synergistic and antagonistic responses of herbicide combinations. Weed Science 15:20-22. and 2) Equation 2 from Huang, Z., and K. A. Falco. 2021. Synergy assessment for plant growth by independent joint action theory. HortScience 56(5):623-626. doi: 10.21273/HORTSC115731-21. A one-sample, two-tailed t-test was used to assess the statistical significance of the difference between the observed and predicted means.

Both methodologies suggested a synergistic effect.

### Method 1: Independent Joint Action (Colby 1967 Percent of Control)

The predicted percent of control according to the Colby method for Independent Joint Action of the combination of 0.36 g ANE+0.30 g Carnitine on NPQt is 68.05. The observed result was 88.83, which yields a synergy ratio of 1.31. This is greater than the predicted result, meaning the combination performed further from 0 than expected.

### Method 2: Independent Joint Action (Huang and Falco 2021 Plant Growth)

The predicted mean according to the Huang and Falco method for Independent Joint Action of the combination of 0.36 g ANE+0.30 g Carnitine on NPQt is 0.28. The observed result was 0.37, which is 30.53% greater than the predicted result.

### Example 7: Soybean Subject to Salinity Stress

Supplementing the efficacy investigations of Examples 2 and 5, a further investigation was performed to assess the effect of seed treatment on mitigating the effects of salinity stress in soybean plants and specifically to investigate evidence for synergistic effect.

The same Seed Treatments solutions (Table 20) and trial protocol were used as in Example 6, but seeds were planted at 1.5″. To coat seeds, 0.3 mL of treatment solution was pipetted into a 500-mL glass beaker around the edges and 30 g of soybean seed was added. Marula soybean seed (reference SEED-003) from Prograin was used, without any other treatment.

### Non-Photochemical Quenchinq

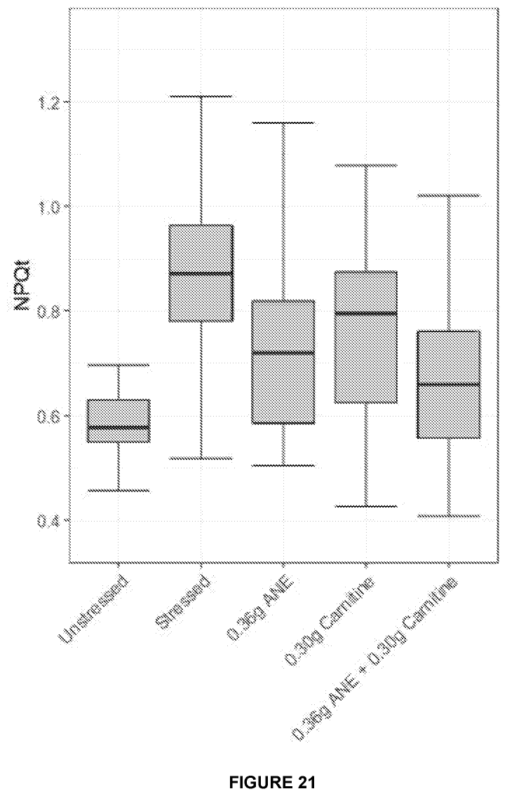

An estimate of NPQt was obtained using the MultispeQ™ (B-4003-AA) from PhotosynQ, according to manufaturer's directions. For the photosynthesis measuments a total of 12-14 plants per treatment were measured. Results are shown in Table 22 and FIG. 21. For Example 7, parametric analysis of variance (ANOVA) was done using the aov function in the ggpubr package, and multiple comparison testing was done with the Tukey test (alpha=0.1) using the TukeyHSD function in the stats package in R.

TABLE 22

| Treatment group | Mean | Difference (%) |
|---|---|---|
| Unstressed | 0.59 | −32.3 |
| Stressed | 0.87 | 0.0 |
| 0.36 g ANE | 0.74 | −14.5 |
| 0.30 g Carnitine | 0.76 | −12.4 |
| 0.36 g ANE + 0.30 g Carnitine | 0.69 | −20.6 |

Synergy was assessed using the two methodologies described in Example 6. Both methodologies suggested a synergistic effect.

### Method 1: Independent Joint Action (Colby 1967 Percent of Control)

The predicted percent of control according to the Colby method for Independent Joint Action of the combination of 0.36 g ANE+0.30 g Carnitine on NPQt is 74.91. The observed result was 79.4, which yields a synergy ratio of 1.06. This is greater than the predicted result, meaning the combination performed further from 0 than expected.

### Method 2: Independent Joint Action (Huang and Falco 2021 Plant Growth)

The predicted mean according to the Huang and Falco method for Independent Joint Action of the combination of 0.36 g ANE+0.30 g Carnitine on NPQt is 0.65. The observed result was 0.69, which is 6% greater than the predicted result.

### Shoot Weight

Shoot weight was evaluated to assess whether the positive effects observed in NPQt was reflected in more robust plant grown. Seven cell packs per treatment were measured for shoot weight. While the sample size was limited the combination showed a positive effect on growth consistent with the positive effects observed in NPQt.

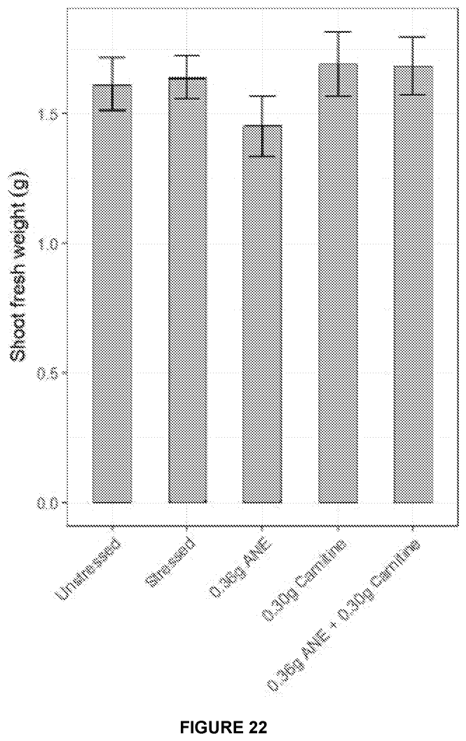

Results are shown in Table 23 and FIG. 22.

TABLE 23

| Treatment | Mean | Difference (%) |
|---|---|---|
| Unstressed | 1.62 | −1.8 |
| Stressed | 1.65 | 0.0 |
| 0.36 g ANE | 1.45 | −11.7 |
| 0.30 g Carnitine | 1.69 | 3.0 |
| 0.36 g ANE + 0.30 g Carnitine | 1.69 | 2.5 |

Synergy was assessed using the two methodologies described in Example 6. Both methodologies suggested a synergistic effect.

### Method 1: Independent Joint Action (Colby 1967 Percent of Control)

The predicted percent of control according to the Colby method for Independent Joint Action of the combination of 0.36 g ANE+0.30 g Carnitine on Shoot Fresh Weight is 90.93. The observed resultwas 102.54, which yields a synergy ratio of 1.13. This is greaterthan the predicted result, meaning the combination performed further from 0 than expected.

### Method 2: Independent Joint Action (Huang and Falco 2021 Plant Growth)

The predicted mean according to the Huang and Falco method for Independent Joint Action of the combination of 0.36 g ANE+0.30 g Carnitine on Shoot Fresh Weight is 1.5. The observed result was 1.69, which is 12.78% greater than the predicted result (P=0.14).

As can be seen therefore, the examples described above and illustrated are intended to be exemplary only. The scope is indicated by the appended claims.

Many further features and combinations thereof concerning the present improvements will appear to those skilled in the art following a reading of the instant disclosure.

1. A method of preventing or mitigating the effects of abiotic stress in a plant comprising administering to the plant, a seed of the plant or to a growth medium of the plant an effective amount of a combination comprising L-carnitine and seaweed extract.

2. The method of claim 1, wherein the method is a seed treatment, and wherein the method comprises administering 50-800 g of L-carnitine per tonne of seed and administering 50-800 g of seaweed extract per tonne of seed.

3. The method of claim 1, wherein the seaweed used to produce the extract is from the class Phaeophyceae.

4. The method of claim 3, wherein the seaweed extract is produced from the species Ascophyllum nodosum.

5. The method of claim 3, wherein the seaweed extract is produced using an alkaline hydrolysis extraction.

6. The method of claim 1, wherein the combination consists or consists essentially of L-carnitine and seaweed extract and, optionally, water.

7. The method of claim 1, wherein mitigating abiotic stress comprises one or more of:
a) increased plant vigour;
b) increased root growth and development;
c) increased shoot growth and development;
d) increased plant growth rate;
e) increased photosynthetic rate and capacity;
f) improved yield;
compared to a control plant that is not administered the combination while subjected to an abiotic stress, or a crop grown under similar environmental conditions that is not administered the combination while encountering abiotic stress.

8. The method of claim 1, wherein the plant is a cereal or legume, optionally, wheat, corn or soybean.

9. A plant biostimulant composition having as active ingredients L-carnitine and a seaweed extract, and optionally water.

10. The composition of claim 9, wherein the seaweed extract is from the class Phaeophyceae.

11. The composition of claim 10, wherein the seaweed extract is from the species Ascophyllum nodosum.

12. The composition of claim 9, wherein the weight ratio of the L-carnitine to the seaweed extract in the composition is between 10:1 and 1:10.

13. The composition of claim 12, wherein the weight ratio of the L-carnitine to the seaweed extract in the composition is between 2:1 to 1:2.

14. The composition of claim 9 containing approximately equals amounts by weight of L-carnitine and the seaweed extract.

15. The composition of claim 9, wherein the composition is an aqueous composition containing 100 to 250 g/L of seaweed extract and 100 to 250 g/L of the L-carnitine.

16. The composition of claim 9, wherein when applied in an effective amount to a plant, a seed of the plant or to a growth medium of the plant, the composition mitigates abiotic stress in the plant, comprising one or more of:
a) increased plant vigour;
b) increased root growth and development;
c) increased shoot growth and development;
d) increased plant growth rate;
e) increased photosynthetic rate and capacity;
f) improved yield;
compared to a control plant that is not administered the composition and is subjected to the same or equivalent abiotic stress as the plant.

17. A seed treatment comprising the composition as defined in claim 9.

18. The composition of claim 9, wherein the L-carnitine and the seaweed extract are both in particulate form.

19. A kit comprising a first package containing L-carnitine and a second package containing seaweed extract and directions for combining the L-carnitine and the seaweed extract to produce an effective amount of a plant biostimulant composition.

20. The kit of claim 19, wherein the plant biostimulant composition is a plant biostimulant composition having as active ingredients L-carnitine and a seaweed extract, and optionally water.
